(19) United States
(12) Patent Application Publication
(10) Pub. No.: US 2025/0017622 A1
(43) Pub. Date: Jan. 16, 2025

(54) INTERVERTEBRAL DISC NUCLEUS PULPOSUS IMPLANT

(71) Applicant: MOVING SPINE AG, ZÜRICH (CH)

(72) Inventors: Robert FRIGG, Bettlach (CH); Jonas WIDMER, Fällanden (CH); Mazda FARSHAD, Zumikon (CH)

(73) Assignee: MOVING SPINE AG, ZÜRICH (CH)

(21) Appl. No.: 18/771,427

(22) Filed: Jul. 12, 2024

Related U.S. Application Data
(60) Provisional application No. 63/526,571, filed on Jul. 13, 2023.

(51) Int. Cl. A61B 17/34; A61F 2/44

(52) U.S. Cl. CPC A61B 17/3468; A61F 2002/444

(57) ABSTRACT

A method of injecting a foldable implant (1) into a body cavity of a patient includes the step of providing the foldable implant (1). The method further includes the step of injecting the provided implant (1) in an unfolded state into the body cavity, wherein in the unfolded state the implant (1) is essentially straight extending along a longitudinal axis (L). The method further includes the step of folding the injected implant (1) from the unfolded state into a folded state, wherein in the folded state the implant (1) has a plurality of loops (2) each extending radially from a fold center (3).

### CROSS-REFERENCE TO RELATED APPLICATIONS

This application claims the benefit of Provisional U.S. Patent Application, Ser. No. 63/526,571, filed 13 Jul. 2023. The co-pending Provisional Application is hereby incorporated by reference herein in its entirety and is made a part hereof, including but not limited to those portions which specifically appear hereinafter.

### BACKGROUND OF THE INVENTION

### Field of the Invention

The present invention relates to a foldable implant for injection into a body cavity of a patient. Further disclosed are a kit for injection of the implant into a body cavity of a patient, a method of preparing the implant and a method of injecting the implant into the body cavity of a patient.

### Discussion of Related Art

The intervertebral disc functions to distribute forces between vertebral bodies, absorb shock and allow movement between adjacent vertebral bodies, thereby stabilizing the spine. An intervertebral disc includes a gelatinous nucleus pulposus, an annulus fibrosis and two vertebral endplates. The nucleus pulposus is surrounded by the annulus fibrosis.

Intervertebral discs are subject to age- and disease-related degeneration and deterioration processes, which lead to a range of unhealthy conditions, including back pain and limited mobility. For example, herniated discs are common, and typically occur when normal wear, or exceptional strain, causes a disc to rupture. Degenerative disc disease typically results from the normal aging process, in which the tissue gradually loses its natural water and elasticity, causing the degenerated disc to shrink and possibly rupture.

Treatment of these conditions often involves surgical removal of a portion or all of the intervertebral disc. As the removal may lead to collapse of the disc space, which impairs the stability of the spine, different implants have been developed to fill an intervertebral disc space following removal of all or part of the intervertebral disc in order to prevent disc space collapse and to promote fusion of adjacent vertebrae surrounding the disc space.

Beyond intervertebral discs and joints, joints such as the trapeziometacarpal joint may be subject to osteoarthritis, which may require operative treatments involving implants. As an example, rhizarthrosis is frequently treated by removing the trapezium bone and stabilizing the base of the thumb by ligament reconstruction. There exists a need to improve the treatment of rhizarthrosis. More broadly, other synovial joints are present in the mammalian appendicular skeleton. A typical synovial joint comprises two bone ends covered by a layer of articular cartilage. As the natural aging process progresses, the cartilage covering the joint may deteriorate and start to fray. Consequently, there exists a need for implants and methods effective for supplementing or replacing the cartilage that lubricates and protects the joint.

### SUMMARY OF THE INVENTION

The known nuclear replacement implants, and implants for injection into body cavities more broadly, suffer from a range of disadvantages. Once positioned in the intervertebral disc space, such as the nucleus pulposus, they may migrate inside the disc space and/or may be expelled from the disc space partially or fully. In particular, many known nuclear replacement implants are injected into the intervertebral disc space and are prone to being expelled from the intervertebral disc space after injection through the opening through which the implant was previously injected.

Furthermore, many of the known implants are not able to adopt different shapes or geometries to occupy differently shaped body cavities. Additionally, the exact shapes and geometries required for different patients may be different and may require patient-specific adaption of the implant, which many known implants are not able to accommodate. Consequently, many known implants do not efficiently occupy the space in the body cavity and therefore do not efficiently transfer the occurring forces between the adjacent vertebrae. In particular, the ability to absorb shock is seriously impaired. Furthermore, certain regions of a body cavity are difficult to reach, in particular when implants are injected into said cavity. As an example, regions orthogonal to the direction of injection may be difficult to reach with an implantable implant.

The lack of efficient occupation of the available space in the body cavity may favor expulsion of the implant and/or migration over time. Both these movements are undesired because they impair the balance of forces acting on the implant and may lead to deterioration of adjacent tissue.

The known implants also possess limited mechanical strength and durability to withstand the significant forces exerted on the implants through the spine. A further disadvantage of some known implants is that they are not suitable for minimally invasive surgery. Furthermore, many known implants are made of a non-biological material, such as an inorganic ceramic material. This precludes the implant to grow together with the adjacent tissue. Furthermore, many known implants never fully return the full range of motion desired.

It is therefore an object of the present disclosure to advance the state of the art with respect to foldable implants, kits for injecting an implant into a body cavity of a patient, method of preparing such implants and methods of injecting an implant into a body cavity of a patient. In particular, it is an object to provide foldable implants, which improve distribution of forces between vertebral bodies and minimize the risk of expulsion or migration once injected inside the body cavity. More broadly, it is an object to provide foldable implants, which contribute to efficient load transmission through the vertebral column and which efficiently absorb shock. It is a further object to provide implants, which may be used to restore the healthy positioning and arrangement of bones and tissues, including restoring a collapsed disc space, for example in the treatment of foraminal stenosis. It is a further object to provide implants, which are suitable for adopting a broad range of shapes and geometries, such that they may efficiently occupy a variety of different body cavities. It is a further object to provide implants, which improve the mobility of the spine, in particular to improve the mobility of the adjacent vertebrae.

According to the present disclosure, these objects are addressed by the features of the independent claims. In addition, further advantageous embodiments follow from the dependent claims and the description.

The present disclosure relates in a first aspect to a foldable implant for injection into a body cavity of a patient. In an unfolded state, the implant is essentially straight extending along a longitudinal axis. In a folded state, the implant has a plurality of cauliflower like loops each extending radially from a fold center. The loops form cushions for load transfer perpendicular to the folding direction.

By providing a plurality of loops each extending radially form a fold center, forces between adjacent intervertebral bodies may be efficiently distributed, which allows for improved load transmission through the vertebral column, thus increasing mobility and decreasing pain. The plurality of loops extending from the fold center essentially act as a load cushion to absorb shocks and distribute forces efficiently. Additionally, the implant may be tailored to the specific requirements of a given body cavity and/or patient with respect to the load-bearing and load-distributing properties. As an example, by varying the geometry, arrangement and shape of the loops, different compression properties may be obtained in different sections of the body cavity and for different force application directions. Thus, complex force distribution profiles may be obtained throughout the entire space of the body cavity.

Additionally, by providing a plurality of loops each extending radially form a fold center, the risk of the implant being expelled from the body cavity, such as the intervertebral disc space, e.g. the nucleus pulposus, is minimized. Meanwhile, by being essentially straight extending along a longitudinal axis in the unfolded state, the implant may be implanted into the body cavity in a minimally invasive fashion. As an example, the implant may be injected along its longitudinal axis into a body cavity, such as the intervertebral disc space. Once inside the body cavity, the implant may adopt its folded state, which typically occupies a large volume inside the body cavity. Additionally, in its folded state, the implant typically has a significant expansion in directions orthogonal to the axis of injection, which allows to efficiently occupy differently shaped body cavities.

The implant may be transformed from its unfolded state into its folded state. Depending on the application, this transformation may be reversible or essentially irreversible. Depending on the application, the implant may be folded on injection into the body cavity or after injection into the body cavity.

The foldable implant is typically suitable for injection into the intervertebral disc space, such as the nucleus pulposus. However, depending on the application, the implant may also be suitable for injection into other body cavities. As an example, the implant may also be used in the treatment of rhizarthrosis.

In its unfolded state, the implant has a proximal end and a distal end opposite the proximal end in longitudinal direction. Similarly, the implant may comprise a main body having a proximal end and a distal end opposite the proximal end in longitudinal direction. The distal end is typically defined as the end that is first injected into the body cavity.

In its folded state, the plurality of loops each extend radially from a fold center. As an example, the plurality of loops may extend in a substantially fan-shaped manner. The two outermost loops of the plurality of loops may define a widening angle. Depending on the application, the widening angle may be at least 30 degrees, preferably at least 45 degrees, more preferably from 60 degrees to 220 degrees. The widening angle is defined as the angle between a first line and a second line intersecting the first line at the fold center, wherein the first line halves the first outermost loop and the second line halves the second outermost loop. It is understood that since the loops are three-dimensional, the first line (respectively the second line) halves the cross-section of the first loop (respectively the second loop). The first outermost loop (respectively the second outermost loop) is the loop corresponding to the most proximal loop section (respectively the most distal loop section) in the unfolded state.

The plurality of loops may, for example, extend in a substantially planar fashion. In other words, the plurality of loops may extend between an upper plane and a lower plane, as outlined above for the implant, wherein the upper plane and the lower plane preferably have a distance from each other of less than 20 mm, preferably less than 10 mm, preferably less than 5 mm.

In the unfolded state, the implant is essentially straight. In other words, the implant may extend significantly further in the longitudinal direction compared to the directions orthogonal to the longitudinal direction. In still other words, the implant may be significantly longer than it is thick. As an example, the implant may in the unfolded state be essentially linear. Depending on the application, the implant may also be slightly curved. As an example, when the implant is made of a biological material such as ligament, it may naturally display slight deviations form a perfectly linear geometry in the unfolded state. It is understood that the implant is configured to be injected into a body cavity through an injection device. As such, it may be the injection device that determines the geometry of the implant in the unfolded state. As an example, the implant may be unstrained or relaxed in the unfolded state. It is also possible that a preloaded implant is held in an essentially straight geometry by an injection device. This is possible because the implant is bendable.

It is understood that the implant is a three-dimensional object. As such, in its unfolded state, the implant may for example extend between an upper plane, a lower plane, a first lateral plane and a second lateral plane, wherein the upper and lower plane are parallel to each other and the first lateral plane and the second lateral plane are parallel to each other and are both orthogonal to the upper plane and to the lower plane. Depending on the application, the distance between the upper and the lower plane and the distance between the first lateral plane and the second lateral plane may not exceed 20 mm, preferably may not exceed 10 mm, preferably may not exceed 5 mm.

In its folded state, the implant has a plurality of loops. Depending on the application, the implant may have less than 20 loops, such as less than ten loops, such as from two loops to ten loops. The loops may differ from other in size and/or shape. The plurality of loops each extend radially from a fold center. The fold center is configured for forming the plurality of loops by bringing the fold positions of the implant into proximity of each other. In the folded state, the fold initiating elements may be in direct or indirect contact with each other. For example, the fold initiating elements may in the folded state be connected with each other, such as connected by a fiber. In the unfolded state, the fiber may, for example, be arranged on a mantle surface of the main body of the implant. In other words, the fiber may be arranged peripherally on a cross-section of the main body of the implant. Depending on the application, each fold initiating element may in the folded state be within 20 mm, preferably within 15 mm, more preferably within 10 mm, more preferably within 5 mm, more preferably within 3 mm, of the other fold initiating elements. The indicated distance may, for example, refer to the minimum distance between two fold initiating elements. Depending on the application, the fold center may have a volume of less than 500 mm³, such as less than 200 mm³, such as less than 100 mm³.

The implant may in the folded state be connected to a vertebral endplate in order to further reduce the risk of migration of the implant. As an example, the fold center may be configured to be connected to a vertebral endplate, such as by means of a suture anchor.

In an embodiment, the implant comprises a plurality of fold initiating elements and a main body having a plurality of fold positions interspaced in the longitudinal direction by loop sections. Each fold position may be connected to a fold initiating element. In the unfolded state, the fold initiating elements may be interspaced from each other in the longitudinal direction. In the folded state, the fold initiating elements may be arranged in proximity of each other to define the fold center, such that for each loop section a corresponding loop is formed.

The main body has a plurality of fold positions, which are interspaced, in the longitudinal direction, by loop sections. In other words, two adjacent fold positions are interspaced in the longitudinal direction by a loop section. Each loop section is configured for forming a loop in the folded state. Typically, each fold position is connected to one fold initiating element. The implant body may, for example, have up to 30 fold positions, preferably up to 20 fold positions, more preferably from three to 15 fold positions. Accordingly, the implant body may, in the folded state, have up to 29 loops, preferably up to 19 loops, more preferably from two to 14 loops. The loop sections may, for example, have a length in longitudinal direction in the unfolded state of up to 40 mm, preferably up to 30 mm, more preferably up to 20 mm, even more preferably from 2 mm to 15 mm. The length of a loop section in longitudinal direction in the unfolded state determines the circumference of the corresponding loop in the folded state.

The fold initiating elements are configured for initiating folding of the main body at the corresponding fold position to which they are connected. Typically, the fold initiating elements are directly connected to the fold positions of the main body. As an example, the fold initiating elements may be arranged peripherally on a cross-section of the main body of the implant. In further embodiments, the fold initiating elements may be offset from a central longitudinal axis of the main body of the implant by at least 0.1 mm, preferably at least 0.5 mm. Depending on the application, the fold initiating elements may be connected to the fold positions by a fiber, a thread, a coil, a suture or a strand. The fold initiating elements may, for example, be made of a synthetic material, such as a polymer.

Different positions of the main body of the implant in longitudinal direction may be selected as fold positions. The main body may have a proximal fold position and a distal fold position. The proximal fold position (respectively distal fold position) is defined as the fold position that, in the unfolded state, is nearest in longitudinal direction to the proximal end (respectively distal end) of the main body. The proximal fold position may, for example, be arranged within 10 mm, preferably within 5 mm, of the proximal end of the main body. Additionally or alternatively, the distal fold position may, for example, be arranged within 10 mm, preferably within 5 mm, of the distal end of the main body. Depending on the application, the proximal fold position and/or the distal fold position may have a distance from the proximal end respectively distal end or at least 2 mm, preferably at least 3 mm. This may reduce the likelihood of detaching of the respective end of the main body from the fold initiating element, in particular after implantation.

The fold initiating elements typically have an influence on how the implant body is folded up and therefore influence the shape of the implant in the folded state. By determining the position of the fold initiating elements, it is possible to influence the folding pattern. As an example, arranging the fold initiating elements peripherally on a cross-section of the main body of the implant may result in a fold center from which each of the loops extend radially. Additionally, by providing the fold initiating elements peripherally on a cross-section of the main body, the mechanical stress on the main body at the fold position during folding may be minimized.

In an embodiment, the fold initiating elements are arranged on an outer surface of the main body of the implant. As an example, in the unfolded state, the fold initiating elements may be arranged on the same face of the main body. This may be used to ensure that in the folded state the plurality of loops extend between an upper plane and a lower plane, wherein the upper plane and the lower plane are parallel to each other and have a distance from each other of less than 20 mm. Depending on the application, the fold initiating elements may be arranged such that they face in the same direction, preferably in a direction essentially orthogonal to the longitudinal direction. As an example, the fold initiating elements may be arranged such that a line connecting the fold initiating elements extends essentially in parallel to the longitudinal direction of the main body of the implant. Preferably, the fold initiating elements are arranged peripherally on and on the same face of a longitudinal cross-section of the main body of the implant. As an example, in embodiments in which the implant main body is cuboid having four mantle surfaces extending in the longitudinal direction, the fold initiating elements may be arranged on the same mantle surface.

In an embodiment, the foldable implant further comprises a fold actuator operatively coupled to the fold initiating elements. The fold actuator is configured for bringing the fold initiating elements into proximity of each other, such that the plurality of loops are formed from the loop sections. The fold actuator may be a mechanical actuator. The fold actuator may, for example, be configured for engaging with the fold initiating elements, e.g. in a form-fit fashion. As an example, the fold actuator may comprise a fiber connecting the fold initiating elements. The fiber may then be used to bring the fold initiating elements into proximity of each other, e.g. by drawing the fold initiating elements together. The fiber may, for example, extend further from a proximal end of the implant main body, such that on injection of the implant, at least a portion of the fiber may be positioned outside the body. This allows a surgeon to control the folding from outside the body.

The fold actuator may comprise a retention structure configured for engaging with the fold initiating elements, e.g. in a form-fit fashion. As an example, the retention structure may be arranged on a distal end of the fiber connected to the fold initiating elements. Depending on the application, the retention structure may, for example, be a bulge in the fiber. The retention structure may have a diameter that is larger than an inner diameter of the fold initiating elements, such that the fold initiating elements are retained by the retention structure. In further embodiments, the retention structure may comprise a hook configured for receiving the fold initiating elements.

The fold initiating elements may be realized in different shapes and/or geometries. As an example, a fold initiating element may comprise a retention element configured for engaging with the retention structure. The retention element may, for example, comprise one or more of a hook, a curve, a coil or a retention loop. The term “retention loop” refers to a loop, wherein the adjective “retention” clarifies that the loop is different from the loops extending radially from the fold center. Depending on the application, the retention loops may in the unfolded state be arranged such that for each retention loop, a plane defined by an opening enclosed by the retention loop extends essentially perpendicularly to the longitudinal direction of the main body of the implant. The retention loop may, for example, be essentially oval, such as circular or elliptical. Depending on the application, the retention loops may have an inner perimeter of up to 6 mm, preferably up to 3 mm.

As mentioned above, the implant may be folded into its folded state during or after injection into the body cavity. Accordingly, depending on the application, the retention structure may be configured for engaging with the fold initiating elements such that, when the implant main body is ejected from an outlet opening of an injection device, the fold initiating elements are retained by the retention structure, such that the interspacing loop sections fold up. As an example, the retention structure may be arranged within a predetermined distance from an outlet opening of an injection device. Depending on the application, the predetermined distance may be up to 30 mm, preferably up to 20 mm, more preferably up to 10 mm. By arranging the retention structure within the predetermined distance from the outlet opening, the position at which the folding of the implant occurs may be determined. As an example, when the retention structure is arranged within 5 mm of an outlet opening of an injection device, the implant main body will be ejected from the outlet opening for at least the first 5 mm in longitudinal direction, until the first fold initiating element engages with and is retained by the retention structure, such that a subsequent loop section begins to fold. Depending on the application, the retention structure may be conntectable to the outlet opening of an injection device.

Depending on the application, the loop sections may have essentially the same or different loop section lengths. A loop section length is defined as the interspacing in longitudinal direction between the adjacent fold positions of the loop section. In some embodiments, at least two loop sections have a different loop section length, such that in the folded state the corresponding loops have different sizes. Different sizes may, for example, be used to achieve different patterns of the implant in the folded state. As an example, the loop sections arranged in the longitudinal direction at both ends of the main body may have a larger loop section length than the other loop sections. This allows for a wide expansion of the implant in a lateral direction in the folded state, which reduces the risk of expulsion of the implant through the hole through which it was injected. Thus, such an arrangement may e.g. be used for body cavities that are wider than they are deep, with respect to the direction along which the implant is injected. In further embodiments, the section lengths of the loop sections may progressively increase from the loop sections arranged in the longitudinal direction at both ends of the main body towards a loop section arranged between these loop sections. This allows for a wide expansion of the implant in the direction of injection. Thus, such an arrangement may be used e.g. for particularly deep body cavities.

Depending on the application, the cross-sectional profile of a loop sections may be essentially unchanged along the longitudinal direction or may vary along the longitudinal direction. As an example, the thickness of a loop section may vary along the longitudinal direction. Additionally, or alternatively, the shape of the cross-section may vary along the longitudinal direction. For example, towards the middle of a loop section in longitudinal direction, a loop section may have a circular cross-section with a small diameter to facilitate deformation, such as bending. Towards the surrounding fold initiating elements, the cross-section may have a larger diameter to enhance the stability of the loop being formed. This exemplary embodiment may allow for a pronounced radial expansion of the loop. More broadly, a varying cross-sectional profile along the longitudinal direction may be used to influence the shape and structure of the loop being formed. In an embodiment, at least one loop section has a cross-sectional profile varying along the longitudinal direction. In an embodiment, at least two loop sections have a different cross-sectional profile along the longitudinal direction, such that in the folded state the corresponding loops have different shapes.

The main body of the implant may have different shapes. Depending on the application, the main body of the implant may have the shape of a strand, string or thread. Depending on the application, the main body may have a length in longitudinal direction of up to 150 mm, preferably up to 100 mm, more preferably up to 50 mm. Additionally or alternatively, depending on the application, the main body may have an average thickness of up to 15 mm, preferably up to 10 mm, more preferably up to 6 mm. Typically, the length of the main body in longitudinal direction is may be significantly higher than the average thickness of the main body. Depending on the application, the length may be at least three times, preferably at least five times, higher than the average thickness.

The main body may have a smaller cross-section at the fold positions than in the loop sections. This may facilitate folding of the main body of the implant at the fold positions and allow for mechanically robust loops.

The cross-section of the main body along the longitudinal direction may be oval, such as circular or elliptical. Depending on the application, the cross-section along the longitudinal direction may also be rectangular or tubular. As an example, in the case of a tubular cross-section, an inner volume of the tube may be filled with a fluid, such as a liquid, which may contain a drug. As outlined above, the cross-section of the main body may vary along the longitudinal direction. Thus, the cross-section of the main body may have multiple of the above-mentioned shapes along the longitudinal direction.

Depending on the application, the main body may have a flexural modulus varying along the longitudinal direction in the unfolded state. The varying flexural modulus may, for example, be achieved by providing different cross-sectional profiles and/or different diameters along the longitudinal direction and/or different materials varying along the longitudinal direction. By varying the flexural modulus along the longitudinal direction, the propensity to and degree of folding may be varied along the longitudinal direction. As an example, in the unfolded state the flexural modulus of the main body at the fold positions may be lower than the average flexural modulus of the main body in the loop sections. By providing a smaller flexural modulus at the fold positions, folding at these positions is facilitated.

For a given position along the longitudinal direction of the main body, the flexural modulus may also be different for different radial bend directions. A different flexural modulus with respect to different radial bend directions may, for example, be achieved by an appropriate cross-sectional profile and/or different materials. Depending on the application, at least one fold position in the unfolded state, the main body may have a flexural modulus with respect to a target bend direction that is smaller than a flexural modulus with respect to a bend direction that is either orthogonal to or opposite to the target bend direction, wherein the target bend direction extends perpendicularly from the center of the longitudinal axis of the main body towards the fold initiating element connected to the fold position. As an example, the main body may have a rectangular cross-section with two shorter sides opposite each other and two longer sides opposite each other. In this case, bending in the direction orthogonal to the longer sides will be mechanically favored over bending the direction orthogonal to the shorter sides.

The flexural modulus, as used herein, is preferably determined using a 3-point bend test in which the force is applied to the position whose flexural modulus is given. The force is typically applied orthogonally to the longitudinal direction.

The implant may be made of various different materials. The implant may comprise an allograft, a xenograft, an autograft or a synthetic composite. As an example, the main body may comprise an allograft, a xenograft, an autograft or a synthetic composite. Depending on the application, the implant may be made of at least 30 wt.-%, preferably at least 50 wt.-%, more preferably at least 70 wt.-%, collagen based on the dry weight of the implant. In an exemplary embodiment, the main body may comprise a collagenous material. The implant may, for example, comprise a ligament or a tendon. In an embodiment, the main body of the implant comprises a ligament or a tendon. Depending on the application, the main body may comprise a reinforcement structure, which may be used to increase mechanical strength. The reinforcement structure may, for example, be integrally connected with the allograft, xenograft, autograft or synthetic composite. Additionally, or alternatively, the reinforcement structure may be helically wrapped around the allograft, xenograft, autograft or synthetic composite.

In a typical embodiment, the body cavity is the intervertebral disc space, preferably the intervertebral disc nucleus pulposus.

In a second aspect, the present disclosure relates to a kit for injecting an implant into a body cavity of a patient. The kit comprises a foldable implant according to any of the embodiments described herein. The kit further comprises an injection device configured for injecting the implant into a body cavity of a patient.

The injection device may, for example, include a syringe having a syringe body and a piston configured for extruding the implant from the syringe body through an opening outlet of the syringe body. The piston is configured for contacting a proximal end of the implant contained inside the syringe body. On ejection of the implant, a distal end of the implant is first ejected from the outlet opening of the syringe body. The injection device is typically essentially straight and typically extends along a longitudinal direction. The injection device is typically tubular and configured for receiving the implant body.

Depending on the application, the kit may comprise a fold actuator operatively coupled to the fold initiating elements of the implant and configured for bringing the fold initiating elements into proximity of each other, such that the plurality of loops are formed from the loop sections. The fold actuator may be releasably connectable to the injection device, preferably releasably connectable to an outlet opening of the injection device. As an example, the fold actuator may be releasably connectable to the outlet opening of the injection device by a connection element, such as a string or a hook. Additionally, or alternatively, the fold actuator may include a fiber connected to the fold initiating elements. The fiber may, for example, protrude from the proximal end of the injection device and may be configured to be actuated by a surgeon performing the injection procedure. As an example, an end of the fiber may be kept outside the body during the minimally invasive injection and, after complete injection of the implant body, the fiber may be pulled in order to bring the fold initiating elements into proximity of each other, such that the plurality of folds are formed.

In further embodiments, the fold actuator may be connected to the outlet opening of the injection device, such that the fold initiating elements are brought into proximity of each other during injection of the implant main body, such that the plurality of folds are formed.

In some embodiments, the kit further comprises a connection element, such as a suture anchor, configured for connecting the implant to a vertebral endplate. As an example, the connection element may be configured for connecting the fold center of the implant to the vertebral endplate.

In a third aspect, the present disclosure relates to a method of preparing an implant according to any one of the embodiments described herein. This method includes providing an essentially straight implant extending along a longitudinal axis, wherein the implant comprises a main body having a plurality of fold positions interspaced in the longitudinal direction by loop sections. The method further includes connecting to at least two, preferably all, of the fold positions a fold initiating element.

The method may further include providing a fold actuator operatively coupled to the fold initiating elements and configured for bringing the fold initiating elements into proximity of each other, such that the plurality of loops are formed from the loop sections.

In a fourth aspect, the present disclosure relates to a method of injecting a foldable implant into a body cavity of a patient. The method comprises the steps of a) providing the foldable implant, preferably an implant according to any one of the embodiments disclosed herein. Typically, the implant is provided in an unfolded state. The method further comprises the step of b) injecting the provided implant in an unfolded state into the body cavity, wherein in the unfolded state the implant is essentially straight extending along a longitudinal axis. It is understood that typically, the implant is injected along the longitudinal axis into the body cavity using an injection device. The method further comprises the step of c) folding the injected implant from the unfolded state into a folded state, wherein in the folded state the implant has a plurality of loops each extending radially from a fold center.

Different body cavities may be envisioned in connection with this method. For example, the body cavity may be the intervertebral disc space, such as the intervertebral disc nucleus pulposus.

The method may for example be used to repair the intervertebral disc nucleus. For example, the nucleus pulposus of a diseased or damaged intervertebral disc may be at least partially replaced by the foldable implant. This method may be used to treat various diseases involving the intervertebral discs, such as foraminal stenosis or spinal disc herniation.

The method disclosed herein typically further includes the step of preparing the body cavity, such as the intervertebral disc space, for injection of the implant. The step of preparing the body cavity for injection is carried out before the injection of the implant. It may be carried out before, simultaneously with or subsequently to the step of providing the foldable implant. The step of preparing the body cavity typically includes providing an opening to the intervertebral disc space, which may include spreading of the body cavity, such as the intervertebral disc space. The opening may also be a tear in the annulus fibrosus, e.g. resulting from disc hernia.

Additionally, or alternatively, the step of preparing the body cavity may also include at least partially clearing the body cavity. This may, for example, include removing at least a portion of the nucleus pulposus or another material present in the respective body cavity. Typically, the amount of material removed is chosen such that sufficient room is created for injection of the implant. More specifically, the amount is preferably chosen such that after insertion and unfolding of the implant, the body cavity reaches target dimensions. For example, the intervertebral disc space may then reach a target disc height. When treating foraminal stenosis, for example, the target disc height may be chosen such that compression of the spinal nerves is avoided.

In some variants, the method further includes fixation of a fixation structure, such as a suture anchor or a bone anchor, in the body cavity. For example, the fixation structure may be fixated in the intervertebral disc space, such as by fixating it to the cranial and/or caudal endplate. Preferably, the fixation structure is fixated such that it is arranged in or near the center of the intervertebral disc space. The step of fixation of the fixation structure is typically carried out before the step of injecting the implant into the body cavity. The step of fixation of the fixation structure may be carried out after removing at least a portion of the nucleus pulposus. In some variants, during the step of folding the injected implant from the unfolded state into the folded state, the implant is folded around the fixation structure, such as around the suture anchor or bone anchor. Thereby, the fixation structure may coincide with the fold center and the loops may extend radially from the fixation structure.

Depending on the application, the injected implant may be connected to a vertebral endplate, e.g. using the fixation structure. For example, the implant may be connected to the vertebral endplate by means of a suture anchor or bone anchor, e.g. through a knot.

Depending on the application, step c) may be carried out essentially simultaneously with or subsequently to step b). Step c) may also be carried out with a slight delay relative to step b). In a variant, in step c) the folding of the implant (1) commences when the implant is either fully or partially injected in the body cavity. The relative order of steps b) and c) may, inter alia, be controlled by arranging a retention structure within a predetermined distance from the outlet opening. As an example, the folding of the implant may commence once the fold initiating elements engage with and are retained by the retention structure. Depending on the predetermined distance, step c) may commence with a delay relative to step b). In an embodiment, step b) comprises positioning a retention structure within 20 mm of an opening outlet of the injection device, such that, when the implant is ejected from the outlet opening, the fold initiating elements are retained by the retention structure, such that the implant is folded according to step c).

In an embodiment, step c) may further include bringing fold initiating elements in proximity of each other to define the fold center. Depending on the application, the step of bringing the fold initiating elements in proximity of each other may be effected by a fold actuator comprising a fiber connected to the fold initiating elements. The method may further include a step d) of binding together at least some, preferably all, of the fold initiating elements. As an example, the fiber connected to the fold initiating elements may be used for binding together the fold initiating elements.

When connecting the implant to a vertebral endplate, that step may for example be carried out simultaneously with or subsequent to step d). The implant may be connected to the vertebral endplate by means of a suture anchor or bone anchor. Connecting the implant to the vertebral endplate may include suturing. In an embodiment, the fold center is connected to the vertebral endplate.

The embodiments disclosed herein in the context of other aspects of the disclosure, such as in particular the first aspect, also apply to all other aspects of the disclosure, particularly to the method of the fourth aspect. Thus, for example, in the folded state the plurality of loops may extend in a substantially fan-shaped manner between an upper plane and a lower plane, wherein the upper plane and the lower plane are parallel to each other and have a distance from each other of less than 20 mm.

In a further variant, the implant may comprise a plurality of fold initiating elements and a main body having a plurality of fold positions interspaced in the longitudinal direction by loop sections, wherein each fold position is connected to a fold initiating element, wherein
a. in the unfolded state the fold initiating elements are interspaced from each other in the longitudinal direction and
b. in the folded state the fold initiating elements are arranged in proximity of each other to define the fold center, such that for each loop section a corresponding loop is formed.

The fold initiating elements may for example be arranged peripherally on a cross-section of the main body of the implant.

In the unfolded state the fold initiating elements may be arranged on the same face of the main body, such that in the folded state the plurality of loops extend between an upper plane and a lower plane, wherein the upper plane and the lower plane are parallel to each other and have a distance from each other of less than 20 mm.

The folding of the implant may be effected in different ways. For example, the implant may comprise a plurality of fold initiating elements and a main body having a plurality of fold positions interspaced in the longitudinal direction by loop sections, wherein each fold position is connected to a fold initiating element. In the unfolded state the fold initiating elements may be interspaced from each other in the longitudinal direction and in the folded state the fold initiating elements may be arranged in proximity of each other to define the fold center, such that for each loop section a corresponding loop is formed. The fold initiating elements may for example be arranged peripherally on a cross-section of the main body of the implant. In the unfolded state the fold initiating elements may be arranged on the same face of the main body, such that in the folded state the plurality of loops extend between an upper plane and a lower plane, wherein the upper plane and the lower plane are parallel to each other and have a distance from each other of less than 20 mm.

Different mechanisms may be envisioned to initiate the folding. For example, step c) may include actuating a fold actuator operatively coupled to the fold initiating elements, wherein the fold actuator is configured on actuation to bring the fold initiating elements into proximity of each other, such that the plurality of loops are formed from the loop sections. The fold actuator may for example comprise a fiber connected to the fold initiating elements, wherein actuating the fold actuator may comprise moving the fiber, preferably pulling the fiber.

In a variant, the fold actuator comprises a retention structure configured for engaging with the fold initiating elements such that, when the main body of the implant is ejected from an outlet opening of an injection device, the fold initiating elements are retained by the retention structure, such that the loop sections interspacing the fold initiating elements fold up.

The loops may have different shapes and geometries and the different shapes and geometries may for example results from variations of the loop sections. For example, at least two loop sections may have a different loop section length, such that in the folded state the corresponding loops have different sizes. Depending on the application, the main body may comprise at least one loop section having a cross-sectional profile varying along the longitudinal direction. Additionally, or alternatively, at least two loop sections may have a different cross-sectional profile along the longitudinal direction, such that in the folded state the corresponding loops have different shapes. In a further variant, at the fold positions the main body may have a smaller cross-section than in the loop sections.

Different material properties may be used to bias or otherwise influence the folding process. For example, in the unfolded state the flexural modulus of the main body at the fold positions may be lower than the average flexural modulus of the main body in the loop sections. It is also possible that at least one fold position in the unfolded state, the main body has a flexural modulus with respect to a target bend direction that is smaller than a flexural modulus with respect to a bend direction that is either orthogonal to or opposite to the target bend direction, wherein the target bend direction extends perpendicularly from the center of the longitudinal axis of the main body towards the fold initiating element connected to the fold position.

Different materials may be used for the implant. The material may be natural or synthetic or a mixture thereof. For example, the implant may comprise an allograft, a xenograft, an autograft or a synthetic composite. Depending on the application, the implant may be made of at least 30 wt.-%, preferably at least 50 wt.-%, more preferably at least 70 wt.-%, collagen based on the dry weight of the implant. Preferably, the implant may comprise a ligament or tendon.

The method of injecting an implant into a body cavity of a patient may also be used in a method of treating a patient in need of an implant for a body cavity. As an example, the patient may be a patient in need of a replacement of the intervertebral disc nucleus pulposus. Thus, the body cavity may be the intervertebral disc space. The intervertebral disc nucleus pulposus may be replaced fully or partially. In an embodiment of the method of treating the patient, the injection device is inserted into the intervertebral space of the patient. As an example, the injection device may be inserted through a tear in the annulus fibrosus caused by the disc hernia. The injection device may be inserted into the body from a dorsal direction.

The patient may also be a patient suffering from rhizarthrosis. The patient may also be a patient suffering from foraminal stenosis. In foraminal stenosis, the lateral nerve is compressed by a narrow foramen, resulting in clinical symptoms. The narrowing of the foramen can be caused by a collapsed disc space where the distance between the upper and lower pedicle is reduced. By inserting an implant, such as an allograft, into the intervertebral space, the foramen can be widened and the nerve root is decompressed.

More generally, the embodiments disclosed herein may be used to repair an intervertebral disc nucleus, which may be damaged or diseased or otherwise harmed. Thus, further described is a method of intervertebral disc nucleus repair comprising a) at least partially clearing an intervertebral disc space of a damaged or diseased intervertebral disc. In a further step b), a foldable implant is injected into the at least partially cleared intervertebral disc space in an unfolded state. In the unfolded state the implant is essentially straight extending along a longitudinal axis. In a further step c), the injected implant is folded from the unfolded state into a folded state, wherein in the folded state the implant has a plurality of loops each extending radially from a fold center.

### BRIEF DESCRIPTION OF SEVERAL VIEWS OF THE DRAWINGS

The invention described herein will be more fully understood from the detailed description given herein below and the accompanying drawings which should not be considered limiting to the invention described in the appended claims. The drawings show:

FIG. 1A shows a foldable implant in its unfolded state;

FIG. 1B shows a foldable implant in its folded state;

FIG. 2 shows a sectional view of a foldable implant in its unfolded state and arranged inside an injection device;

FIG. 3 shows a sectional view of a foldable implant that is in the process of being ejected from an injection device and in a partially folded state;

FIG. 4 shows a foldable implant in its folded state inside the nucleus pulposus of an intervertebral disc;

FIG. 5 shows a variation of a foldable implant in its unfolded state in a perspective view;

FIG. 6 shows variations of cross-sections of the implant; and

FIG. 7 shows a variation of a method of intervertebral disc nucleus repair.

### DETAILED DESCRIPTION OF THE INVENTION

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

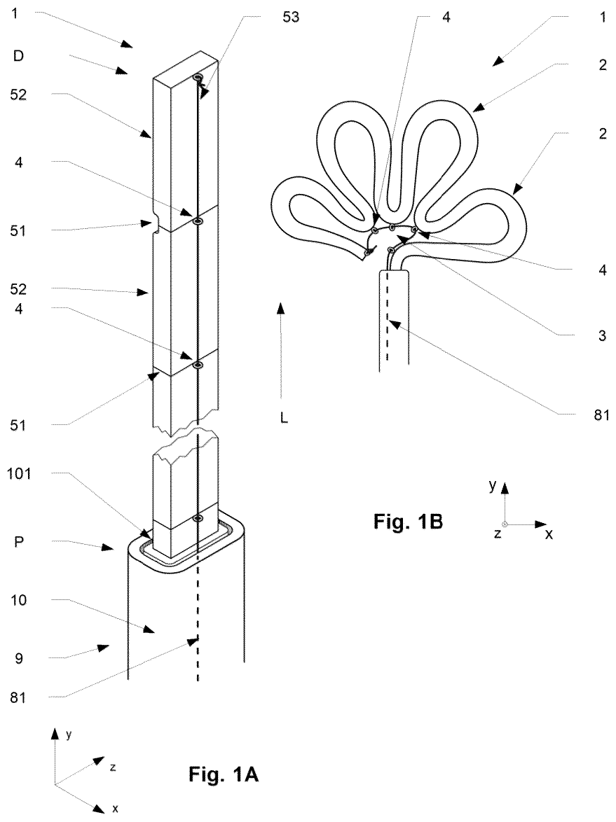

FIG. 1 shows a variation of the foldable implant according to the disclosure. The implant 1 is shown in an unfolded state in perspective view (FIG. 1A) and in a folded state from a top view (FIG. 1B). The implant 1 is shown as it is being ejected from an injection device 10 using a kit 9 disclosed herein.

With reference to the unfolded state (FIG. 1A), the implant 1 is essentially straight and extends in a longitudinal direction L (y-axis). The implant 1 has an essentially rectangular cross-section, which defines a mantle surface having two larger surfaces opposite each other and two smaller surfaces opposite each other. Along the longitudinal direction, the implant 1 has a plurality of fold positions 51 which are interspaced by loop sections 52. At each fold positions 51, the main body is connected to a fold initiating element 4 that has the shape of a loop. The fold initiating elements 4 are all arranged on the same face of the main body, which ensures that the implant is folded in an essentially planar fashion. Furthermore, the fold initiating elements 4 are arranged on one of the larger mantle surfaces, which facilitates folding of the implant 1 because the implant is folded in a direction parallel to the smaller mantle surfaces in which the flexural modulus is lower.

With reference to the folded state (FIG. 1B), the implant 1 has four loops 2, each of which extends radially from a fold center 3. The implant 1 is essentially fan-shaped. The fold center 3 is defined by the fold initiating elements 4, which have been brought into proximity of each other. A fiber 81 is connected to all of the fold initiating elements and acts as a fold actuator 8 to effect folding of the implant 1.

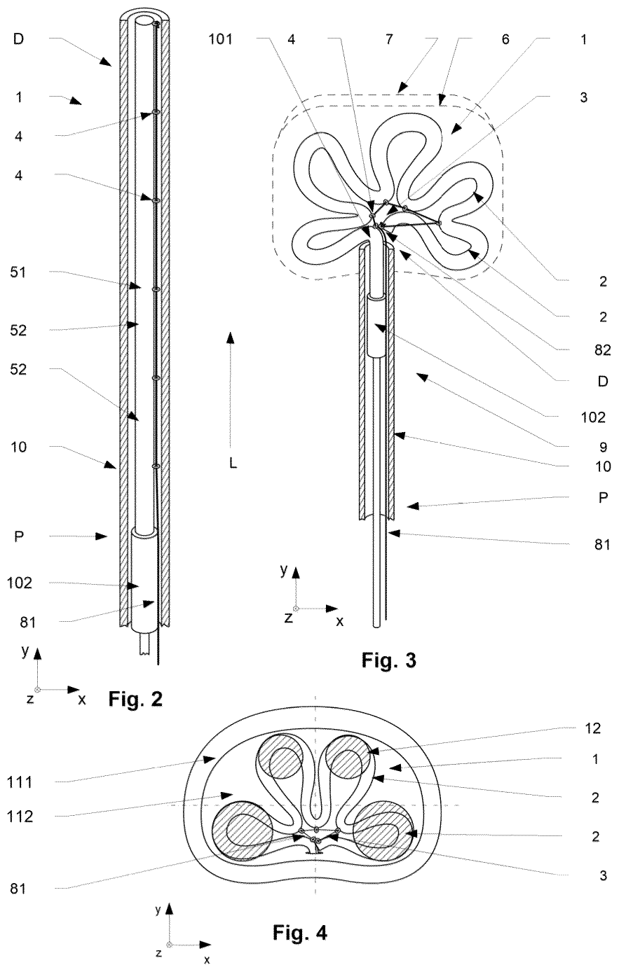

FIG. 2 shows a sectional view of a variation of the implant 1 of the present disclosure, which has a cylindrical cross-section. The implant 1 as shown in the figure is arranged inside an injection device 10 of the kit disclosed herein. The implant 1 also extends in a longitudinal direction L (y-axis) and comprises a main body 5 having five fold positions 51, which are interspaced in the longitudinal direction by four loop sections 52. The main body 5 has a distal end D and a proximal end P. In the variation shown, one fold initiating element 4 is arranged on the distal end D of the main body 5. Also shown is a piston 102 of the injection device 10 and the fiber 81.

FIG. 3 shows the variation of the implant 1 of FIG. 2 as it is being ejected from an outlet opening 101 of the injection device 10. The implant 1 shown in FIG. 3 is in a partially folded state. Depending on the application, the partially folded state may be a target state. In other variations, the fold initiating elements 4 may be brought into even closer proximity to reach a fully folded state as a target state. This may be effected by pulling the fiber 81 that passes through the fold initiating elements 4. As shown, the fiber 81 protrudes from the proximal end P of the injection device 10, such that it may be pulled by a surgeon carrying out the injection process.

FIG. 4 shows an implant 1 in a folded state inside the nucleus pulposus 112 from a top view. By virtue of the four loops 2 which extend radially from the fold center 3, the space of the nucleus pulposus 112 is effectively occupied by the implant 1. The implant 1 in the folded state also has a considerable expansion in the lateral direction (x-axis), which minimizes the risk of migration of the implant 1 or even expulsion of the implant 1 out of the nucleus pulposus. FIG. 4 illustrates that each of the loops 2 forms a cushion for load transfer 12, as illustrated by the hatched circles. As show, the cushions for load transfer are configured for absorbing shocks and contribute to load transmission through the vertebral column.

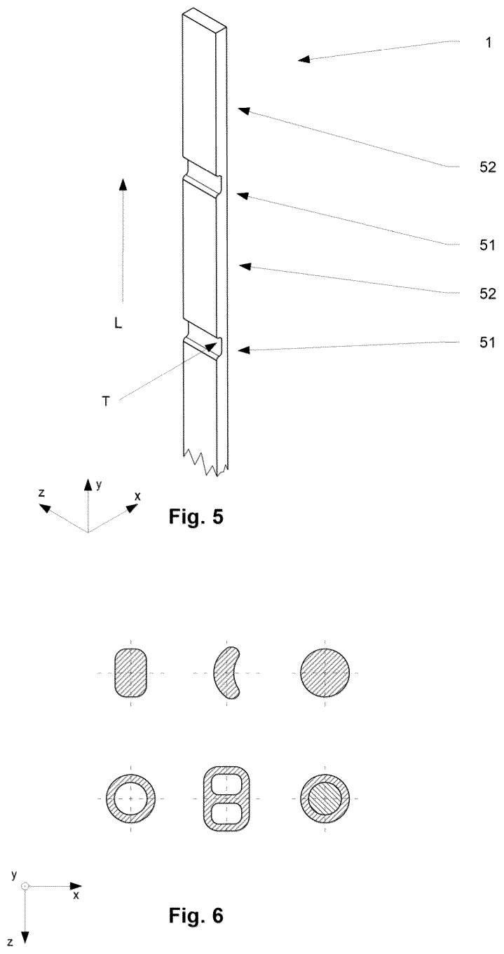

FIG. 5 shows another variation of the implant 1 of the present disclosure in an unfolded state. The implant 1 extends along a longitudinal direction L (y-axis). Only a part of the implant 1 in longitudinal direction is shown. The implant 1 comprises a main body 5 having fold positions 51 and loop sections 52. At the fold positions 51, the cross-section of the implant is smaller than in the loop sections 52. This facilitates folding of the main body 5 at the fold positions 51. Additionally, the cross-section at the fold positions 51 defines a target bend direction T in which the flexural modulus is lowest at the fold position 51.

FIG. 6 shows the cross-section of different variations of the main body 5 of the implant 1. The implants 1 differ in the cross-section of the main body 5, which may be rectangular with rounded edges (top left), sickle-shaped (top middle), essentially circular (top right) or essentially tubular (bottom left and bottom right). In the case of a tubular cross-section, an inner volume of the tube may either be empty (bottom left) or filled, e.g. filled with a fluid (bottom right). Additionally, or alternatively, the cross-section may be essentially eight-shaped, comprising two superimposed tubes (bottom middle). The different cross-sections may be used to favor folding in a specific direction.

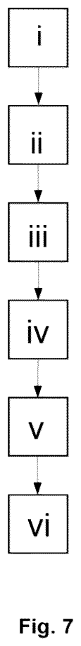

FIG. 7 illustrates a method of intervertebral disc nucleus repair. The method includes injection of a foldable implant into the intervertebral disc space of a patient. In the embodiment discussed, the patient has an impaired intervertebral disc, such as a diseased or damaged intervertebral disc.

The method includes, as a first step i), providing a foldable implant according to any of the embodiments disclosed herein. In a further step ii), the intervertebral disc space is cleared by removing at least some of the nucleus pulposus from the intervertebral disc space. In a further step iii), a bone anchor is fixated in the cranial and/or caudal endplate. The bone anchor will serve as a fixation point for the implant to be injected.

In a further step iv), the implant is injected into the intervertebral disc space in an unfolded state in which it is essentially straight and extends along a longitudinal axis. The implant is typically injected through a cannula. A tear in the intervertebral disc may serve as an opening to inject the implant. Depending on the application, the intervertebral disc may also be spread prior to injection of the implant.

In a further step v), the implant is folded from the unfolded state into a folded state, in which it has a plurality of loops each extending radially from a fold center. The fold center may be arranged near the bone anchor.

In a further step vi), the implant may be fixated to the bone anchor by a knot.

1. A method of injecting a foldable implant (1) into a body cavity of a patient, wherein the method comprises the steps of:
a. providing the foldable implant (1);
b. injecting the provided implant (1) in an unfolded state into the body cavity, wherein in the unfolded state the implant (1) is essentially straight extending along a longitudinal axis (L); and
c. folding the injected implant (1) from the unfolded state into a folded state, wherein in the folded state the implant (1) has a plurality of loops (2) each extending radially from a fold center (3).

2. The method according to claim 1, wherein the body cavity is the intervertebral disc space, such as the intervertebral disc nucleus pulposus (112).

3. The method according to claim 1, further comprising connecting the injected implant (1) to a vertebral endplate.

4. The method according to claim 3, wherein the implant (1) is connected to the vertebral endplate by means of a suture anchor or bone anchor.

5. The method according to claim 1, wherein in the folded state the plurality of loops (2) extend in a substantially fan-shaped manner between an upper plane (6) and a lower plane (7), wherein the upper plane (6) and the lower plane (7) are parallel to each other and have a distance from each other of less than 20 mm.

6. The method according to claim 1, wherein the implant (1) comprises a plurality of fold initiating elements (4) and a main body (5) having a plurality of fold positions (51) interspaced in the longitudinal direction (L) by loop sections (52), wherein each fold position (51) is connected to a fold initiating element (4), wherein
a. in the unfolded state the fold initiating elements (4) are interspaced from each other in the longitudinal direction (L) and
b. in the folded state the fold initiating elements (4) are arranged in proximity of each other to define the fold center (3), such that for each loop section (52) a corresponding loop (2) is formed.

7. The method according to claim 6, wherein the fold initiating elements (4) are arranged peripherally on a cross-section of the main body (5) of the implant (1).

8. The method according to claim 6, wherein in the unfolded state the fold initiating elements (4) are arranged on the same face of the main body (5), such that in the folded state the plurality of loops (2) extend between an upper plane and a lower plane, wherein the upper plane and the lower plane are parallel to each other and have a distance from each other of less than 20 mm.

9. The method according to claim 6, wherein step c) includes actuating a fold actuator (8) operatively coupled to the fold initiating elements (4), wherein the fold actuator (8) is configured on actuation to bring the fold initiating elements (4) into proximity of each other, such that the plurality of loops (2) are formed from the loop sections (52).

10. The method according to claim 9, wherein the fold actuator (8) comprises a fiber (81) connected to the fold initiating elements (4), wherein actuating the fold actuator (8) comprises moving the fiber (81), preferably pulling the fiber (81).

11. The method according to claim 9, wherein the fold actuator (8) comprises a retention structure (82) configured for engaging with the fold initiating elements (4) such that, when the main body (5) of the implant (1) is ejected from an outlet opening (91) of an injection device (9), the fold initiating elements (4) are retained by the retention structure (82), such that the loop sections (52) interspacing the fold initiating elements (4) fold up.

12. The method according to claim 5, wherein at least two loop sections (52) have a different loop section length, such that in the folded state the corresponding loops (2) have different sizes.

13. The method according to claim 6, wherein the main body comprises at least one loop section (52) having a cross-sectional profile varying along the longitudinal direction (L).

14. The method according to claim 6, wherein at least two loop sections (52) have a different cross-sectional profile along the longitudinal direction (L), such that in the folded state the corresponding loops (2) have different shapes.

15. The method according to claim 6, wherein at the fold positions (51) the main body (5) has a smaller cross-section than in the loop sections (52).

16. The method according to claim 6, wherein in the unfolded state the flexural modulus of the main body (5) at the fold positions (51) is lower than the average flexural modulus of the main body (5) in the loop sections (52).

17. The method according to claim 6, wherein at least one fold position (51) in the unfolded state, the main body (5) has a flexural modulus with respect to a target bend direction (T) that is smaller than a flexural modulus with respect to a bend direction that is either orthogonal to or opposite to the target bend direction (T), wherein the target bend direction (T) extends perpendicularly from the center of the longitudinal axis (L) of the main body (5) towards the fold initiating element (4) connected to the fold position (51).

18. The method according to claim 1, wherein the implant (1) comprises one of an allograft, a xenograft, an autograft, and a synthetic composite.

19. The method according to claim 1, wherein the implant (1) is made of at least 30 wt.-%, preferably at least 50 wt.-%, more preferably at least 70 wt.-%, collagen based on the dry weight of the implant.

20. The method according to claim 1, wherein the implant (1) comprises a ligament or tendon.

21. The method according to claim 1, wherein in step c) the folding of the implant (1) commences when the implant is either fully or partially injected in the body cavity.

22. A method of intervertebral disc nucleus repair comprising:
a. at least partially clearing an intervertebral disc space of a damaged or diseased intervertebral disc;
b. injecting into the at least partially cleared intervertebral disc space a foldable implant (1) in an unfolded state, wherein in the unfolded state the implant (1) is essentially straight extending along a longitudinal axis (L);
c. folding the injected implant (1) from the unfolded state into a folded state, wherein in the folded state the implant (1) has a plurality of loops (2) each extending radially from a fold center (3).
